(19) United States
(12) Patent Application Publication
(10) Pub. No.: US 2012/0310250 A1
(43) Pub. Date: Dec. 6, 2012

(54) DEVICE FOR ASSISTING CESAREAN DELIVERIES

(75) Inventors: Brigid Kathleen McCue, Hanover, MA (US); Matthew McCue Nonnamaker, Hanover, MA (US)

(73) Assignee: Tufts Medical Center, Inc., Boston, MA (US)

(21) Appl. No.: 13/483,257

(22) Filed: May 30, 2012

Related U.S. Application Data
(60) Provisional application No. 61/493,620, filed on Jun. 6, 2011.

(51) Int. Cl. A61B 17/44

(52) U.S. Cl. 606/121

(57) ABSTRACT

A catheter device for facilitating release of a structure lodged within a body cavity includes a flexible catheter and carrier configured to mount the catheter to a finger. The catheter includes a leading end having an opening configured to permit fluids within the catheter to exit the catheter, and a proximal end configured to connect to a fluid source. A method using the catheter device to release a fetal head from a maternal pelvis during cesarean delivery of the fetus is provided.

### CROSS REFERENCE TO RELATED APPLICATIONS

This application claims the benefit of U.S. Provisional Application No. 61/493,620, titled “Device for Assisting Cesarean Deliveries” filed Jun. 6, 2011. The contents of the above application are incorporated herein by reference.

### BACKGROUND OF THE INVENTION

The obstetrical challenge of the cesarean delivery after a long labor often includes difficulty in safely dislodging the impacted fetal head. While the occurrence of fetal cervical fracture during cesarean delivery is rare, fetal skull fracture and intracerebral hemorrhage, trauma to maternal tissues resulting in hemorrhage and bladder damage, and injury to the delivering obstetrician is not. As protracted labors and cesarean section rates rise, a simple but effective device to facilitate delivery is needed.

### SUMMARY

In some aspects, a device is provided for facilitating release of a structure lodged within a body cavity. The device includes a flexible catheter having a leading end, the leading end including an opening configured to permit fluids within the catheter to exit the catheter, and a carrier adjoining the leading end of the catheter and configured to mount the catheter to a finger.

The device may include one or more of the following features: The flexible catheter further includes an internal fluid passage that extends from the leading end to an opposed end, and the opening communicates with the fluid passage. The flexible catheter further includes a body extending between the leading end and an opposed end, the body being tubular in shape including a tube width and a tube height that are the same in dimension, and the leading end having a width that is greater than the tube width and a height that is less than the tube height. The leading end includes a base that is wide relative to the width of the body, and a terminal end extending from the base, the width of the terminal end tapering from the base to an apex. The carrier is disposed on a side of the leading end. The leading end is generally flat on a side opposed to the carrier. The leading end flat side includes a raised pattern configured to enhance frictional engagement of the leading end flat side with a surface of the structure. The opening is disposed on a side of the leading end opposed to the carrier. The opening is triangular. The area defined by the periphery of the opening is greater than the cross-sectional area of the fluid passageway. The device is formed of silicon. The carrier is configured to surround at least the distal phalange of a finger. The carrier is configured to surround the distal phalange and middle phalange of a finger. The carrier is elastic. The opposed end of the catheter is configured to be connected to a fluid source. The opposed end of the catheter includes a connector having a thread configured to engage a corresponding thread on a fluid source.

In some aspects, a device is provided for facilitating release of a structure lodged within a body cavity. The device includes a flexible body, the body having a fluid passageway, a first opening configured to receive a finger, a second opening configured to provide a fluid inlet to the passageway, and a third opening configured to permit fluids within the passageway to exit the body.

The device may include one or more of the following features: The fluid passageway extends from the second opening to the third opening, and the first opening is isolated from the fluid passage. The body includes a tubular portion and has a tube width and a tube height that are the same dimension, the tubular portion extending between a proximal end that includes the second opening and an opposed leading end that includes the third opening, the leading end having a width that is greater than the tube width and a height that is less than the tube height. The leading end includes a base that is wide relative to the width of the body, and a terminal end extending from the base, the width of the terminal end tapering from the base to an apex. The first opening is disposed on a side of the leading end. The leading end is generally flat on a side opposed to the first opening. The flat side of the leading end includes a raised pattern configured to enhance frictional engagement of the flat side with a surface of the structure. The third opening is disposed on a side of the leading end opposed to the first opening. The third opening is triangular. The area defined by the periphery of the third opening is greater than a cross-sectional area of the fluid passageway. The body is formed of silicon. The first opening is configured to surround at least the distal phalange of a finger. The first opening is configured to surround the distal phalange and middle phalange of a finger. The portion of the body defining the first opening is elastic. The proximal end is configured to be connected to a fluid source. The proximal end includes a connector having a thread configured to engage a corresponding thread on a fluid source.

In some aspects, a method of forming a device for facilitating release of a structure lodged within a body cavity is provided. The method includes providing a flexible catheter including a leading end, the leading end including an opening configured to permit fluids within the catheter to exit the catheter, and securing a carrier, configured to mount the catheter to a finger, to the catheter leading end.

In some aspects, a method releasing a fetal head from the pelvis during cesarean delivery of the fetus is provided. The method includes insertion of a catheter into the pelvis via an abdominal incision so that a leading end of the catheter is disposed between the fetal head and the pelvis, injection of a fluid through the catheter into pelvis between the fetal head and the pelvis so as to facilitate release of the fetal head from the pelvis. In some embodiments, the catheter includes a finger carrier and the catheter is finger-mounted during insertion.

A catheter is disclosed that advantageously facilitates release of a structure lodged within a body cavity by inserting a sterile irrigant between the structure and the body cavity so as to break a vacuum holding the structure within the body cavity. For example, in a cesarean delivery, the catheter is placed below the vertex at the time of delivery so as to provide a sterile irrigant between the fetal head and the maternal pelvis. Doing so breaks the vacuum holding the fetal head in the maternal pelvis and lavages the vertex upwards, permitting the practitioner to easily remove the fetal head from the pelvis, and complete the cesarean delivery.

In certain embodiments, the catheter includes a finger carrier disposed adjacent the leading end of the catheter, permitting the catheter to be finger mounted. This configuration further advantageously permits finger-directed placement of the catheter in the body cavity, allowing proper placement despite tight space requirements.

In certain embodiments, the catheter leading end is relatively wide and thin relative to the mid-section and proximal end of the catheter, facilitating insertion of the leading end into tight spaces between closely engaged structures.

In certain embodiments, the leading end is generally flat on a side opposed to the finger carrier. The flat side is provided with surface features configured to enhance the ability of the practitioner to engage the structure to be dislodged from the body cavity. For example, the flat side may include raised ridges configured to enhance the user's ability to grip the fetal scalp while withdrawing the fetal skull from the maternal pelvis. In addition, by providing improved finger grip, less finger flexion force is required to achieve an adequate grip, further reducing trauma to the fetal skull.

Modes for carrying out the present invention are explained below by reference to an embodiment of the present invention shown in the attached drawings. The above-mentioned object, other objects, characteristics and advantages of the present invention will become apparent from the detailed description of the embodiment of the invention presented below in conjunction with the attached drawings.

### BRIEF DESCRIPTION OF THE DRAWINGS

FIG. 1 is a schematic illustration of the catheter device being used to facilitate release of a fetal head impacted within a pelvic cavity during a cesarean delivery.

FIG. 2 is a perspective view of the catheter device of FIG. 1.

FIG. 3 is a perspective view of the tip portion of the catheter device of FIG. 1.

FIG. 4 is a sectional view of the tubular portion as seen across line 4-4 of FIG. 3.

FIGS. 5-7 are alternative embodiments of the tip portion as seen when viewed in the direction of arrow 7 of FIG. 3.

FIG. 8 is a sectional view of the tip portion as seen across line 5-5 of FIG. 3.

FIG. 9 is a sectional view of the tip portion as seen across line 6-6 of FIG. 3.

### DETAILED DESCRIPTION

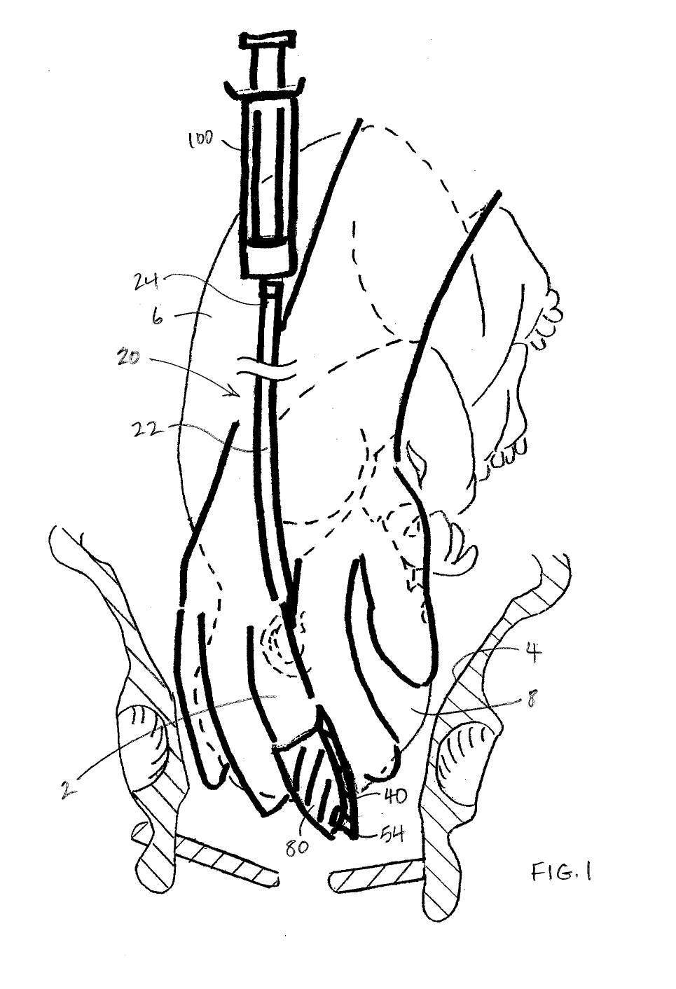

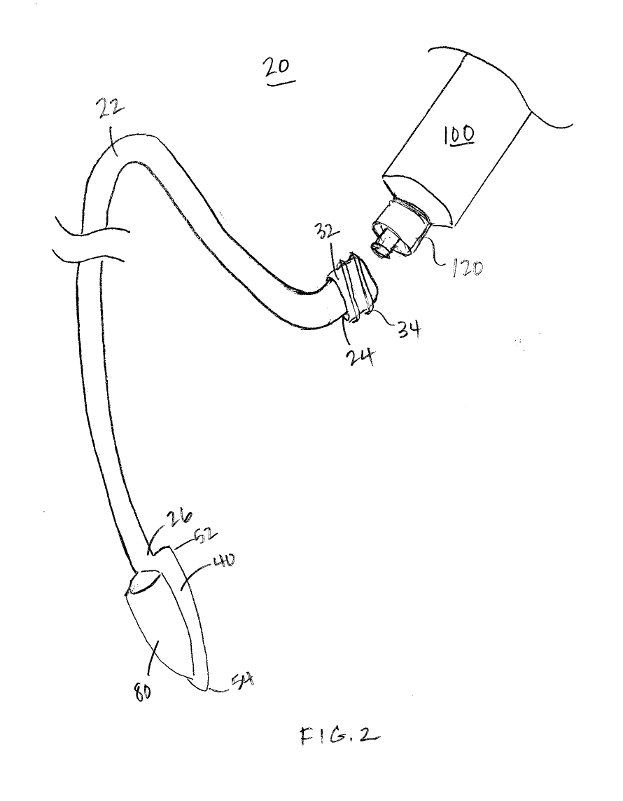

Referring now to FIGS. 1-3, the catheter device 20 includes a tubular portion 22 including a proximal end 24 configured to be secured to a fluid supply 100. The catheter device 20 includes a tip portion 40 that extends from a distal end 26 of the tubular portion, where the distal end 26 is opposed to the proximal end 24. In addition, an internal fluid passageway 30 extends from the proximal end 24 to a terminus 54 of the tip portion 40. As will be discussed in greater detail below, the tip portion 40 has a shape that is wide and flat relative to the tubular portion 22 to facilitate insertion of the catheter device 20 into tight spaces, and particularly between an object 8 and a body cavity surface 4 within which the object 8 is lodged. In addition, the tip portion 40 includes a carrier 80 so that the catheter device 20 can be mounted on, and directed by, a finger 2 during use, as also discussed further below.

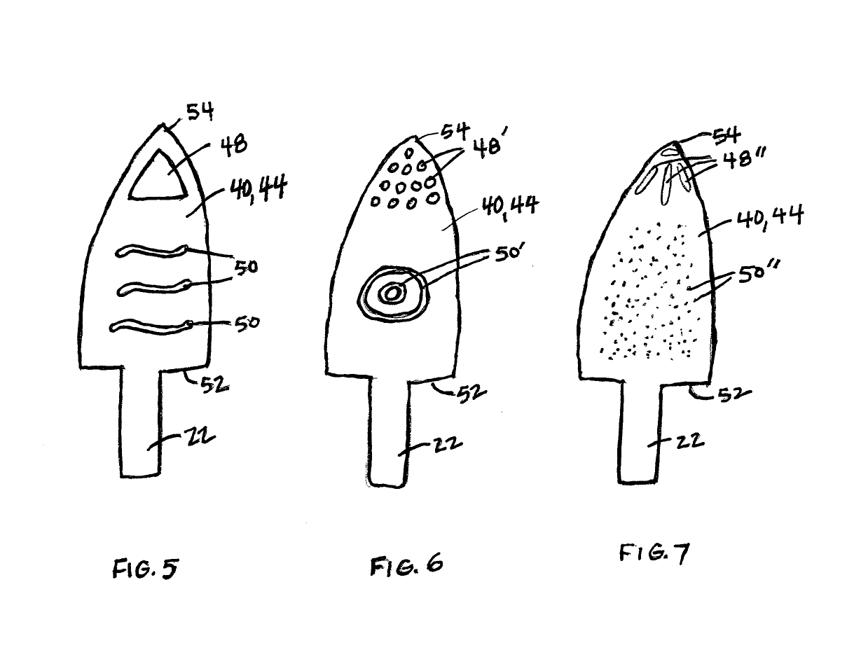

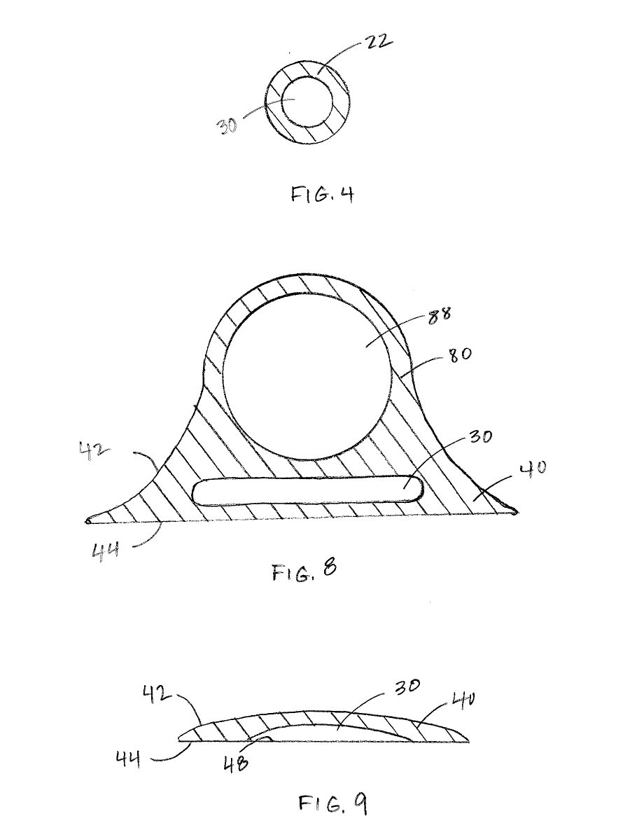

Referring to FIG. 4, the tubular portion 22 has a circular cross-sectional shape, whereby the height and width the section are of equal dimension. The passageway 30 extends coaxially with respect to a longitudinal centerline of the tubular portion 22, and is also circular in cross-sectional shape.

The tubular portion 22 is long relative to its cross-sectional diameter, which may be in a range of 0.1 inches to 0.4 inches. In particular, the tubular portion has sufficient length to permit the user to insert the tip portion 40 within a body cavity while the fluid supply 100 remains outside the body. In some embodiments, the tubular portion has a length of about 20 inches to allow the device 20 to extend from a finger tip to at least an elbow of the user.

Referring again to FIGS. 1-3, in the illustrated embodiment, the fluid supply 100 is a manual syringe having a luer lock syringe tip 102. The connector 32 provided on the tubular portion proximal end 24 includes the corresponding structure, including external threads 34, for engaging the luer lock tip 102. The locking engagement between the connector 32 and the fluid supply 100 is advantageous since it ensures a firm connection between the catheter device 20 and the fluid supply 100 even when fluid pressures generated by the fluid supply are relatively high, as may be required to expel fluid from the catheter tip portion 40 when the device 20 is used in a tight space.

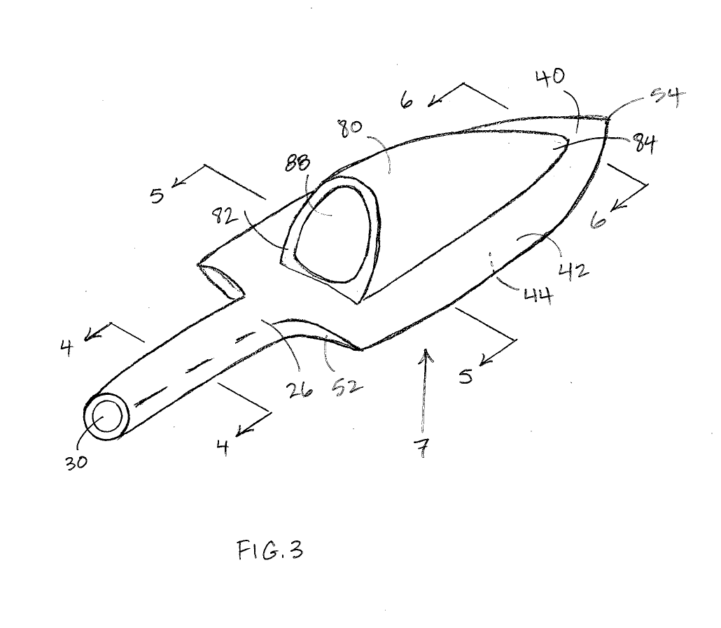

The tip portion 40 extends integrally from the distal end 26 of the tubular portion 22. The tip portion 40 is wide and flat relative to the tubular portion 22, so that it has a width that is greater than the tube width and a height that is less than the tube height. The tip portion 40 includes a dorsal surface 42 which supports the carrier 80, and a ventral surface 44 opposed to the dorsal surface 42. As viewed in the direction of the arrow 7 of FIG. 3, the tip portion 40 has a peripheral shape in the general form of a spade. That is, the tip portion 40 includes a wide base 52 that adjoins the tubular portion distal end 26, and gradually reduces in width along an axial direction, so that the tip portion 40 terminates at the pointed apex 54. Providing the tip portion 40 with a small, pointed leading end is advantageous for initiating insertion of the tip portion 40 into tight, confined spaces.

Referring to FIG. 5, the ventral surface 44 is a generally planar surface and includes an opening 48 that extends through the surface 44 and communicates with the fluid passageway 30. The opening 48 provides an outlet for the fluids supplied by the fluid supply 100 and has an area that is relatively large compared to the cross sectional area of the fluid passageway 30. In the illustrated embodiment, the opening 48 is triangular in shape and an apex of the opening 48 is located at the tip portion apex 54. In addition, the portions of the ventral surface 44 surrounding the opening 48 are smooth to facilitate insertion of the tip portion 40 into tight, confined spaces.

The region of the ventral surface 44 between the opening 48 and that base 52 further includes surface features that enhance frictional engagement of the ventral surface 44 with a surface of the structure to be dislodged. In the illustrated embodiment, the surface features include an array of generally parallel, spaced-apart, protruding ridges 50 that extend generally in the width direction of the tip portion 40. In the case of a cesarean delivery, where the fetal scalp 8 may be coated with vernix caseosa and bodily fluids, the ridges 50 improve the user's ability to grip the fetal skull 8. By improving the user's ability to grip the skull 8, less finger flexion force is required to hold the fetal skull 8 and/or move the fetal skull 8 relative to the maternal pelvis 4.

Referring to FIGS. 6 and 7, alternative surface features can be used to enhance frictional engagement by the ventral surface 44 of the catheter tip portion 40. For example, the array of parallel spaced ridges 50 can be replaced by an array of spaced ridges in the form of concentric rings 50′ (FIG. 6). In another example, the surface features may include providing a set of small, randomly raised dots 50″ (FIG. 7).

Referring to FIGS. 3 and 8, the carrier 80 is disposed on the dorsal surface 42 of the tip portion 40, and is a hollow, elastic, resilient, generally tubular structure. The carrier 80 has an open end 82 adjacent the base 52, and a closed end 84 adjacent to the apex 54. The carrier closed end 84 is formed so as to taper toward the dorsal surface 42, to facilitate insertion of the tip portion into tight spaces. The hollow interior space 88 is dimensioned to receive a portion of a finger, and the carrier length, defined as the distance between the open end 82 and the closed end 84, is sufficient to surround at least the distal phalange of an adult finger 2. In some embodiments, the carrier length is sufficient to surround at least the distal phalange and middle phalange of an adult finger 2. The cross-sectional dimension of the carrier 80 is slightly less than an adult finger 2 so that in use, the carrier 80 is radially stretched when finger-mounted so that friction force and carrier elasticity securely retain the carrier 80 on the finger 2.

The tubular portion 22 and the tip portion 40 of the catheter device 20 are formed of a polymer such as silicone or urethane. In the illustrated embodiment, the carrier 80 is integrally formed as a single unit with the tip portion 40 and tubular portion 20. It is understood, however, that the carrier 80 and/or tip portion 40 may be formed individually and assembled to the tubular portion 22. Coatings may be applied to the device outer surface at at least some locations to improve surface friction properties. For example, at some locations, a lubricant may be applied to reduce device surface friction and enhance the insertability of the device 20 into tight spaces. At other locations, an adhesive may be applied to improve localized gripability.

In use, the catheter device 20 facilitates release of a structure lodged within a body cavity by inserting a sterile irrigant between the structure and the body cavity so as to break a vacuum holding the structure within the body cavity. For example, in a cesarean delivery where the fetal head 8 is lodged within a maternal pelvis, the catheter device is mounted on a finger 2, for example the middle finger, of a user so that the tip portion 40 resides on a palmar side of the finger 2. Specifically, the catheter device 20 is oriented relative to the finger 2 so that palmar surface of the finger 2 overlies the tip portion dorsal surface 42, and the tip portion ventral surface 42 faces away from the palmar surface of the finger 2. In this configuration, the ventral surface 42 can confront the fetal skull 8. The catheter device 20, including the tip portion 40 mounted on the user's finger 2, is inserted into the maternal pelvis via an abdominal incision so that the tip portion 40 is disposed between the fetal head 8 and the pelvis 4, and at least the proximal end 24 of the tubular portion 22 and the fluid supply 100 resides externally relative to the abdominal incision. In some embodiments, the tip portion 40 is advanced to the fetal skull vertex 10. Once the tip portion 40 is in position, sterile fluid is injected through the catheter fluid passageway 30 and exits from the catheter device 20 via the opening 48 into pelvis between the fetal head 8 and the pelvis 4 so as to facilitate release of the fetal head from the pelvis, for example by relieving a region of low pressure between the maternal pelvis 4 and fetal head 8. If necessary, the fluid may be pressurized to facilitate sufficient flow quantities and/or rates. The injected fluid breaks the vacuum holding the fetal head in the pelvis and lavages the vertex upwards. Once the vacuum is released, the user's finger tips, including the finger 2 carrying the catheter device 20, are flexed to withdraw the fetal head 8 from the pelvis 4.

In the illustrated embodiment, the catheter device 20 is described for use in cesarean deliveries to facilitate the release of an impacted fetal head. It is understood, however, that the device may be used to facilitate release of other structures lodged within body cavities. For example, the catheter device may be used to facilitate release of a pessary.

Although in the illustrated embodiment, the tubular portion 22 and fluid passageway 30 each have a circular cross-sectional shape, it is understood that the tubular portion 22 of the catheter device 20 is not limited to this shape. For example, the tubular portion 22 and/or fluid passageway 30 may have polygonal or oval cross-sectional shapes.

In the illustrated embodiment, the fluid supply 100 is a manual syringe having a luer lock syringe tip. However, the fluid supply 100 is not limited to this type of syringe, and may be replaced with, for example, a syringe having different syringe tips. Moreover, the fluid supply 100 is not limited to a syringe, and may be replaced with system including a pump connected to a fluid reservoir.

In the illustrated embodiment the carrier 80 is shaped and dimensioned for mounting on a user's finger. The carrier is not limited to this configuration. For example, the carrier may be in the form of a glove worn on a user's hand, with the catheter leading end 40 secured to a palmar side of a glove finger.

Although in the illustrated embodiment, the opening 48 in the tip portion 40 is a single opening that is triangular in shape, it is understood that the opening 48 is not limited to this configuration. For example, an alternative configuration may include an array of small circular openings 48′ arranged in the vicinity of the apex 54 (see FIG. 6). In another example, another alternative configuration may include plural elongated, mutually-diverging openings 48″ arranged in the vicinity of the apex 54 (see FIG. 7).

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art. Moreover, while a working example of the present invention has been described above, the present invention is not limited to the working example described above, but various design alterations may be carried out without departing from the present invention as set forth in the claims.

What is claimed is:

1. A device for facilitating release of a structure lodged within a body cavity, the device including
a flexible catheter including a leading end, the leading end including an opening configured to permit fluids within the catheter to exit the catheter, and
a carrier adjoining the leading end of the catheter and configured to mount the catheter to a finger.

2. The device of claim 1 wherein the flexible catheter further includes an internal fluid passage that extends from the leading end to an opposed end, and the opening communicates with the fluid passage.

3. The device of claim 1 wherein the flexible catheter further includes a body extending between the leading end and an opposed end,
the body being tubular in shape including a tube width and a tube height that are the same in dimension, and
the leading end having a width that is greater than the tube width and a height that is less than the tube height.

4. The device of claim 1 wherein the leading end includes a base that is wide relative to the width of the body, and a terminal end extending from the base, the width of the terminal end tapering from the base to an apex.

5. The device of claim 1 wherein the carrier is disposed on a side of the leading end.

6. The device of claim 1 wherein the leading end is generally flat on a side opposed to the carrier.

7. The device of claim 6 wherein the leading end flat side includes a raised pattern configured to enhance frictional engagement of the leading end flat side with a surface of the structure.

8. The device of claim 1 wherein the opening is disposed on a side of the leading end opposed to the carrier.

9. The device of claim 1 wherein the opening is triangular.

10. The device of claim 1 wherein the area defined by the periphery of the opening is greater than the cross-sectional area of the fluid passageway.

11. The device of claim 1 wherein the device is formed of silicon.

12. The device of claim 1 wherein the carrier is configured to surround at least the distal phalange of a finger.

13. The device of claim 1 wherein the carrier is configured to surround the distal phalange and middle phalange of a finger.

14. The device of claim 1 wherein the carrier is elastic.

15. The device of claim 2 wherein the opposed end of the catheter is configured to be connected to a fluid source.

16. The device of claim 2 wherein the opposed end of the catheter includes a connector having a thread configured to engage a corresponding thread on a fluid source.

17. A device for facilitating release of a structure lodged within a body cavity, the device comprising
a flexible body, the body including
a fluid passageway,
a first opening configured to receive a finger,
a second opening configured to provide a fluid inlet to the passageway, and
a third opening configured to permit fluids within the passageway to exit the body.

18. The device of claim 17 wherein the fluid passageway extends from the second opening to the third opening, and the first opening is isolated from the fluid passage.

19. The device of claim 17 wherein the body includes a tubular portion and has a tube width and a tube height that are the same dimension, the tubular portion extending between a proximal end that includes the second opening and an opposed leading end that includes the third opening, the leading end having a width that is greater than the tube width and a height that is less than the tube height.

20. The device of claim 19 wherein the leading end includes a base that is wide relative to the width of the body, and a terminal end extending from the base, the width of the terminal end tapering from the base to an apex.

21. The device of claim 19 wherein the first opening is disposed on a side of the leading end.

22. The device of claim 19 wherein the leading end is generally flat on a side opposed to the first opening.

23. The device of claim 22 wherein the leading end flat side includes a raised pattern configured to enhance frictional engagement of the leading end flat side with a surface of the structure.

24. The device of claim 19 wherein the third opening is disposed on a side of the leading end opposed to the first opening.

25. The device of claim 17 wherein the third opening is triangular.

26. The device of claim 17 wherein the area defined by the periphery of the third opening is greater than a cross-sectional area of the fluid passageway.

27. The device of claim 17 wherein the body is formed of silicon.

28. The device of claim 17 wherein the first opening is configured to surround at least the distal phalange of a finger.

29. The device of claim 17 wherein the first opening is configured to surround the distal phalange and middle phalange of a finger.

30. The device of claim 17 wherein the portion of the body defining the first opening is elastic.

31. The device of claim 19 wherein the proximal end is configured to be connected to a fluid source.

32. The device of claim 19 wherein the proximal end includes a connector having a thread configured to engage a corresponding thread on a fluid source.

33. A method of forming a device for facilitating release of a structure lodged within a body cavity, the method including
providing a flexible catheter including a leading end, the leading end including an opening configured to permit fluids within the catheter to exit the catheter, and
securing a carrier, configured to mount the catheter to a finger, to the catheter leading end.

34. A method of releasing a fetal head from the pelvis during cesarean delivery of the fetus, the method including
insertion of a catheter into the pelvis via an abdominal incision so that a leading end of the catheter is disposed between the fetal head and the pelvis,
injection of a fluid through the catheter into pelvis between the fetal head and the pelvis so as to facilitate release of the fetal head from the pelvis.

35. The method of claim 34 wherein the catheter includes a finger carrier and the catheter is finger-mounted during insertion.
